(19) United States
(12) Patent Application Publication
(10) Pub. No.: US 2026/0324697 A1
(43) Pub. Date: Oct. 22, 2026

(54) MATERIALS AND METHODS FOR MASKING BITTERNESS

(71) Applicant: UNIVERSITY OF FLORIDA RESEARCH FOUNDATION, INCORPORATED, Gainesville, FL (US)

(72) Inventors: Yu Wang, Gainesville, FL (US); Jinpyo An, Gainesville, FL (US)

(21) Appl. No.: 19/483,728

(22) PCT Filed: May 9, 2024
(86) PCT No.: PCT/US2024/028553

§ 371 Date: Nov. 12, 2025

Related U.S. Application Data
(60) Provisional application No. 63/467,708, filed on May 19, 2023.

(51) Int. Cl. A23L 27/10; A23L 27/00; A23L 27/12; C07C 41/36

(52) U.S. Cl. CPC A23L 27/11; A23L 27/12; A23L 27/84; A23L 27/86; C07C 41/36

(57) ABSTRACT

A method of masking bitter taste of an ingestible composition comprising adding a bitter-masking compound to the ingestible composition, and a method of obtaining (2fi)-3-(4-hydroxy-3-methoxyphenyl) propane-1,2-diol from allspice via extraction.

### FIELD

This disclosure relates to masking bitter taste of an ingestible composition. More particularly, this disclosure relates to extraction and isolation of compounds that are effective at masking bitter taste of an ingestible composition and to addition of a bitter-masking compound to an ingestible composition to reduce the degree of perceived bitterness of the composition.

### BACKGROUND

Natural aversion to bitter taste can be a deterrent to consuming food items, medications, or other ingestible compositions. Because some compounds that can provide a health benefit can also impart bitter flavor to food or drink items, aversion to bitterness can lead to reduced intake of healthy food items or medications. For instance, many vegetables contain flavonoids, terpenes, or polyphenolic acids, which are generally beneficial to human health but which can impart bitter flavor. Reducing the perceived bitterness of ingestible compositions can remove a deterrent to consuming healthy food items or medications.

Methods for reducing the degree of bitterness of an ingestible composition by removing a bitter component can have disadvantages. For instance, the process of removing a bitter component from a food or drink item can also remove other desirable components. Naringin, a flavanone-7-O-glycoside that is abundant in the membranes and albedo of grapefruit, imparts bitter taste to grapefruit juice. Naringin can be removed from grapefruit juice by treating the juice with a resin that can adsorb naringin; however, adsorptive resins are generally non-selective, and resin adsorption can also remove beneficial secondary metabolites from the juice. Decaffeination processes are commonly used to decrease the content of caffeine in coffee or tea; however, many decaffeination processes use organic solvents which are toxic, environmentally unfriendly, and/or expensive. Furthermore, the approach of removing one or more bitter components from a composition cannot be applied to compositions in which a bitter component is critical to the use of the composition, such as a medication comprising an active component that imparts bitter taste.

Attempting to cover bitter taste with another taste or flavor can also have disadvantages. For instance, bitterness of an ingestible composition can be covered up by adding a component that enhances a different taste sensation, such as sweetness or saltiness. However, adding sugar or salt to an ingestible composition is generally considered unhealthy, and adding sufficient sugar or salt to cover bitter taste may also undesirably alter the flavor profile of a food item. Adding fatty compounds to an ingestible composition may cover up bitter taste, but fatty compounds are also considered unhealthy.

Accordingly, there is a need in the art to provide a method for masking bitter taste of an ingestible composition without undesirably altering health benefits, flavor properties, or other aspects of the composition.

### SUMMARY

One aspect of the disclosure provides method of masking bitter taste of an ingestible composition, the method comprising adding a bitter-masking compound to the ingestible composition, wherein the bitter-masking compound comprises a compound according to Formula (I)

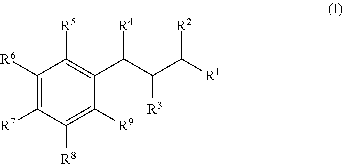

wherein R¹, R², R³, R₄, R⁵, R⁶, R⁷, R⁸ and R⁹ are independently H, CH₃, CH₂CH₃, 1-propyl, 2-propyl, butyl, pentyl, OH, OCH₃, OCH₂CH₃, 1-propyloxy, 2-propyloxy, C₁₋₃alkylene-OH, C₁₋₃alkylene-OCH₃, acyl, substituted acyl, aryl, or substituted aryl.

Another aspect of the disclosure provides a method of obtaining (2R)-3-(4-hydroxy-3-methoxyphenyl) propane-1,2-diol from allspice, the method comprising (a) admixing allspice with an extraction solvent to form a crude mixture having a liquid phase and a solid phase, wherein the extraction solvent comprises a mixture of water and ethanol, containing from about 70 wt. % to about 90 wt. % ethanol based on the total weight of the extraction solvent; and (b) collecting a first portion of the liquid phase, wherein the first portion of the liquid phase would elute from a liquid chromatography column using a first elution solution comprising water and ethanol containing about 20 wt. % to about 40 wt. % ethanol based on the total weight of the first elution solution, wherein the first portion of the liquid phase comprises (2R)-3-(4-hydroxy-3-methoxyphenyl) propane-1,2-diol.

For the methods described herein, optional features, including but not limited to components, compositional ranges thereof, substituents, conditions, and steps are contemplated to be selected from the various aspects, embodiments, and examples provided herein.

Further aspects and advantages will be apparent to those of ordinary skill in the art from a review of the following detailed description, taken in conjunction with the drawings. While the methods described herein are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

For further facilitating the understanding of the present invention, two drawing figures are appended hereto.

FIG. 1 shows the structures of Compounds 1-5.

FIG. 2 shows the results of molecular docking analysis of the bitter receptor site of TAS2R14.

### DETAILED DESCRIPTION

The disclosure provides a method of masking bitter taste of an ingestible composition. The method can include adding a bitter-masking compound to an ingestible composition in an amount sufficient to reduce the perceived bitter taste of the composition. The methods of the disclosure can provide one or more advantages including, but not limited to, reducing the perceived bitterness of an ingestible composition without altering other sensory perceptions (e.g., sweetness or saltiness) of the ingestible composition.

“Comprising” as used herein means that various components, ingredients, or steps that can be conjointly employed in practicing the present disclosure. Accordingly, the term “comprising” encompasses the more restrictive terms “consisting essentially of” and “consisting of.” The present compositions can comprise, consist essentially of, or consist of any of the required and optional elements disclosed herein. The invention illustratively disclosed herein suitably may be practiced in the absence of any element or step which is not specifically disclosed herein.

### Bitter Masking Compounds

The bitter-masking compound comprises a compound according to Formula (I)

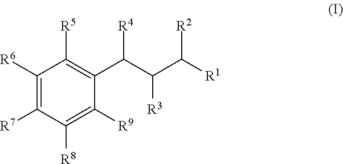

wherein R¹, R², R³, R⁴, R⁵, R⁶, R⁷, R⁸ and R⁹ are independently H, CH₃, CH₂CH₃, 1-propyl, 2-propyl, butyl, pentyl, OH, OCH₃, OCH₂CH₃, 1-propyloxy, 2-propyloxy, C₁₋₃alkylene-OH, C₁₋₃alkylene-OCH₃, acyl, substituted acyl, aryl, or substituted aryl. In embodiments, R¹ and R³ are independently H, OH, OCH₃, C₁₋₃alkylene-OH, or C₁₋₃alkylene-OCH₃, R², R⁴, R⁶, R⁷, and R⁸ are independently H, OH, or OCH₃, and R⁵ and R⁹ are H. In structures shown herein, hydrogen atoms are shown where necessary for clarity. Any hydrogen atom not shown can be considered implied where necessary to provide a full valence shell for, e.g., a central carbon atom.

In general, R¹ can be any small substituent. As used herein, and unless defined otherwise, the term “small” when referring to a substituent refers to a substituent wherein the longest chain (branched or straight) comprises 6 or less atoms. In embodiments, R¹ can be hydrophilic. In embodiments, R¹ can be selected from H, OH, OCH₃, C₁₋₃alkylene-OH, or C₁₋₃alkylene-OCH₃. In embodiments, R¹ is selected from OH, C₁₋₃alkylene-OH, and C₁₋₃alkylene-OCH₃. As used herein, the term “alkylene” refers to linear and branched divalent hydrocarbon groups. The term Chalkylene indicates that the alkylene groups has n carbon atoms. For example, Csalkylene refers to an alkylene group having 3 carbon atoms, such as —(CH₂)₃—, and C₁₋₃alkylene refers to alkylene groups having 1, 2, or 3 carbon atoms. “Me” and “CH₃” are used interchangeably herein to denote a methyl group. In embodiments, R¹ is selected from OH, OCH₃, and CH₂OH. In embodiments, R¹ is OH. In embodiments, R¹ is OCH₃. In embodiments, R¹ is CH₂OH. In embodiments, R¹ can be hydrophobic. In embodiments, R¹ can be CH₃, CH₂CH₃, 1-propyl, 2-propyl, butyl, or pentyl. In embodiments, R¹ can be CH₃. In embodiments, R¹ can be CH₂CH₃. In embodiments, R¹ can be 1-propyl. In embodiments, R¹ can be 2-propyl. In embodiments, R¹ can be butyl. In embodiments, R¹ can be pentyl.

In general, R² can be any small substituent. In embodiments, R² can be hydrophilic. In embodiments, R² can be H, OH, or OCH₃. In embodiments, R² is H. In embodiments, R² is OH. In embodiments, R² is OCH₃. In embodiments, R¹ is OH and R² is H. In embodiments, R¹ is H and R² is OH. In embodiments, both R¹ and R² are H. In embodiments, R¹ is H and R² is OCH₃. In embodiments, R¹ is OCH₃ and R² is H. In embodiments, R¹ is CH₂OH and R² is H. In embodiments, R² can be hydrophobic. In embodiments, R² can be CH₃, CH₂CH₃, 1-propyl, 2-propyl, butyl, or pentyl. In embodiments, R² can be CH₃. In embodiments, R² can be CH₂CH₃. In embodiments, R² can be 1-propyl. In embodiments, R² can be 2-propyl. In embodiments, R² can be butyl. In embodiments, R² can be pentyl.

In general, R³ can be any small substituent. In embodiments, R³ can be hydrophilic. In embodiments, R³ can be selected from H, OH, OCH₃, C₁₋₃alkylene-OH, or C₁₋₃alkylene-OCH₃. In embodiments, R³ is selected from OH, C₁₋₃alkylene-OH, and C₁₋₃alkylene-OCH₃. In embodiments, R³ is selected from OH, OCH₃, and CH₂OH. In embodiments, R³ is OH. In embodiments, R³ is OCH₃. In embodiments, R³ is CH₂OH. In some embodiments, R¹ and R³ are the same. In some embodiments, R¹ and R³ are both OH. In some embodiments, R¹ is H and R³ is OH. In some embodiments, R¹ is H, R² is H, and R³ is OH. In some embodiments, R¹ is OH, R² is H, and R³ is OH. In some embodiments, R¹ is H, R² is OH, and R³ is OH. In some embodiments, R¹ and R³ are both CH₂OH. In some embodiments, R¹ and R³ are both OCH₃. In embodiments, R³ can be hydrophobic. In embodiments, R³ can be CH₃, CH₂CH₃, 1-propyl, 2-propyl, butyl, or pentyl. In embodiments, R³ can be CH₃. In embodiments, R³ can be CH₂CH₃. In embodiments, R³ can be 1-propyl. In embodiments, R³ can be 2-propyl. In embodiments, R³ can be butyl. In embodiments, R³ can be pentyl.

In general, R⁴ can be any small substituent. In embodiments, R⁴ can be hydrophilic. In embodiments, R⁴ can be H, OH, or OCH₃. In embodiments, R⁴ is H. In embodiments, R⁴ is OH. In embodiments, R⁴ is OCH₃. In embodiments, R⁴ and R² can be the same. In embodiments, R² and R⁴ can be different. In embodiments, R² and R⁴ are H. In embodiments, R⁴ and R² are OH. In embodiments, R¹ is OH, R² is H, R³ is OH, and R⁴ is H. In embodiments, R¹ is H, R² is OH, R³ is OH, and R⁴ is H. In embodiments, R¹ is OCH₃, R² is H, R³ is OH, and R⁴ is H. In embodiments, R¹ is OH, R² is H, R³ is OCH₃, and R⁴ is H. In embodiments, R¹ is CH₂OH, R² is H, R³ is OH, and R⁴ is H. In embodiments, R¹ is CH₂OH, R² is H, R³ is OCH₃, and R⁴ is H. In embodiments, R¹ is CH₂OH, R² is H, R³ is CH₂OH, and R⁴ is H. In embodiments, R¹ is OH, R² is H, R³ is CH₂OH, and R⁴ is H. In embodiments, R¹ is OCH₃, R² is H, R³ is CH₂OH, and R⁴ is H. In embodiments, R⁴ can be hydrophobic. In embodiments, R⁴ can be CH₃, CH₂CH₃, 1-propyl, 2-propyl, butyl, or pentyl. In embodiments, R⁴ can be CH₃. In embodiments, R⁴ can be CH₂CH₃. In embodiments, R⁴ can be 1-propyl. In embodiments, R⁴ can be 2-propyl. In embodiments, R⁴ can be butyl. In embodiments, R⁴ can be pentyl.

In general, R⁵ can be any small substituent. Without intending to be bound by theory, it is believed that the presence of certain small substituents on the phenyl ring of the compound according to Formula (I) can improve the binding of the compound to binding sites of bitterness receptors, such as TAS2R¹⁴. In embodiments, R⁵ can be hydrophilic. In embodiments, R⁵ can be H, OH, or OCH₃. In embodiments, R⁵ is H. In embodiments, R⁵ is OH. In embodiments, R⁵ is OCH₃. In embodiments, R⁵ can be hydrophobic. In embodiments, R⁵ can be CH₃, CH₂CH₃, 1-propyl, 2-propyl, butyl, or pentyl. In embodiments, R⁵ can be CH₃. In embodiments, R⁵ can be CH₂CH₃. In embodiments, R⁵ can be 1-propyl. In embodiments, R⁵ can be 2-propyl. In embodiments, R⁵ can be butyl. In embodiments, R⁵ can be pentyl.

In general, R⁶ can be any small substituent. Without intending to be bound by theory, it is believed that the presence of certain small substituents on the phenyl ring of the compound according to Formula (I) can improve the binding of the compound to binding sites of bitterness receptors, such as TAS2R¹⁴. In embodiments, R⁶ can be hydrophilic. In embodiments, R⁶ can be H, OH, or OCH₃. In embodiments, R⁶ is H. In embodiments, R⁶ is OH. In embodiments, R⁶ is OCH₃. In embodiments, R¹ is OH, R² is H, R³ is OH, R⁴ is H, and R⁶ is OCH₃. In embodiments, R¹ is H, R² is OH, R³ is OH, R⁴ is H, and R⁶ is OCH₃. In embodiments, R¹ is OCH₃, R² is H, R³ is OH, R⁴ is H, and R⁶ is OCH₃. In embodiments, R¹ is OH, R² is H, R³ is OCH₃, R⁴ is H, and R⁶ is OCH₃. In embodiments, R¹ is CH₂OH, R² is H, R³ is OH, R⁴ is H, and R⁶ is OCH₃. In embodiments, R¹ is CH₂OH, R² is H, R³ is OCH₃, R⁴ is H, and R⁶ is OCH₃. In embodiments, R¹ is CH₂OH, R² is H, R³ is CH₂OH, R⁴ is H, and R⁶ is OCH₃. In embodiments, R¹ is OH, R² is H, R³ is CH₂OH, R⁴ is H, and R⁶ is OCH₃. In embodiments, R¹ is OCH₃, R² is H, R³ is CH₂OH, R⁴ is H, and R⁶ is OCH₃. In embodiments, R¹ is OH, R² is H, R³ is OH, R⁴ is H, and R⁶ is OH. In embodiments, R¹ is H, R² is OH, R³ is OH, R⁴ is H, and R⁶ is OH. In embodiments, R¹ is OCH₃, R² is H, R³ is OH, R⁴ is H, and R⁶ is OH. In embodiments, R¹ is OH, R² is H, R³ is OCH₃, R⁴ is H, and R⁶ is OH. In embodiments, R¹ is CH₂OH, R² is H, R³ is OH, R⁴ is H, and R⁶ is OH. In embodiments, R¹ is CH₂OH, R² is H, R³ is OCH₃, R⁴ is H, and R⁶ is OH. In embodiments, R¹ is CH₂OH, R² is H, R³ is CH₂OH, R⁴ is H, and R⁶ is OH. In embodiments, R¹ is OH, R² is H, R³ is CH₂OH, R⁴ is H, and R⁶ is OH. In embodiments, R¹ is OCH₃, R² is H, R³ is CH₂OH, R⁴ is H, and R⁶ is OH. In embodiments, R⁶ can be hydrophobic. In embodiments, R⁶ can be CH₃, CH₂CH₃, 1-propyl, 2-propyl, butyl, or pentyl. In embodiments, R⁶ can be CH₃. In embodiments, R⁶ can be CH₂CH₃. In embodiments, R⁶ can be 1-propyl. In embodiments, R⁶ can be 2-propyl. In embodiments, R⁶ can be butyl. In embodiments, R⁶ can be pentyl.

In general, R⁷ can be any small substituent. Without intending to be bound by theory, it is believed that the presence of certain small substituents on the phenyl ring of the compound according to Formula (I) can improve the binding of the compound to binding sites of bitterness receptors, such as TAS2R14. In embodiments, R⁷ can be hydrophilic. In embodiments, R⁷ can be H, OH, or OCH₃. In embodiments, R⁷ is H. In embodiments, R⁷ is OH. In embodiments, R⁷ is OCH₃. In embodiments, R¹ is OH, R² is H, R³ is OH, R⁴ is H, R⁶ is OCH₃, and R⁷ is OH. In embodiments, R¹ is H, R² is OH, R³ is OH, R⁴ is H, R⁶ is OCH₃, and R⁷ is OH. In embodiments, R¹ is OCH₃, R² is H, R³ is OH, R⁴ is H, R⁶ is OCH₃, and R⁷ is OH. In embodiments, R¹ is OH, R² is H, R³ is OCH₃, R⁴ is H, R⁶ is OCH₃, and R⁷ is OH. In embodiments, R¹ is CH₂OH, R² is H, R³ is OH, R⁴ is H, R⁶ is OCH₃, and R⁷ is OH. In embodiments, R¹ is CH₂OH, R² is H, R³ is OCH₃, R⁴ is H, R⁶ is OCH₃, and R⁷ is OH. In embodiments, R¹ is CH₂OH, R² is H, R³ is CH₂OH, R⁴ is H, R⁶ is OCH₃, and R⁷ is OH. In embodiments, R¹ is OH, R² is H, R³ is CH₂OH, R⁴ is H, R⁶ is OCH₃, and R⁷ is OH. In embodiments, R¹ is OCH₃, R² is H, R³ is CH₂OH, R⁴ is H, R⁶ is OCH₃, and R⁷ is OH. In embodiments, R¹ is OH, R² is H, R³ is OH, R⁴ is H, R⁶ is OH, and R⁷ is OH. In embodiments, R¹ is H, R² is OH, R³ is OH, R⁴ is H, R⁶ is OH, and R⁷ is OH. In embodiments, R¹ is OCH₃, R² is H, R³ is OH, R⁴ is H, R⁶ is OH, and R⁷ is OH. In embodiments, R¹ is OH, R² is H, R³ is OCH₃, R⁴ is H, R⁶ is OH, and R⁷ is OH. In embodiments, R¹ is CH₂OH, R² is H, R³ is OH, R⁴ is H, R⁶ is OH, and R⁷ is OH. In embodiments, R¹ is CH₂OH, R² is H, R³ is OCH₃, R⁴ is H, R⁶ is OH, and R⁷ is OH. In embodiments, R¹ is CH₂OH, R² is H, R³ is CH₂OH, R⁴ is H, R⁶ is OH, and R⁷ is OH. In embodiments, R¹ is OH, R² is H, R³ is CH₂OH, R⁴ is H, R⁶ is OH, and R⁷ is OH. In embodiments, R¹ is OCH₃, R² is H, R³ is CH₂OH, R⁴ is H, R⁶ is OH, and R⁷ is OH. In embodiments, R¹ is H, R² is OH, R³ is OH, R⁴ is H, R⁶ is OCH₃, and R⁷ is OCH₃. In embodiments, R¹ is OCH₃, R² is H, R³ is OH, R⁴ is H, R⁶ is OCH₃, and R⁷ is OCH₃. In embodiments, R¹ is OH, R² is H, R³ is OCH₃, R⁴ is H, R⁶ is OCH₃, and R⁷ is OCH₃. In embodiments, R¹ is CH₂OH, R² is H, R³ is OH, R⁴ is H, R⁶ is OCH₃, and R⁷ is OCH₃. In embodiments, R¹ is CH₂OH, R² is H, R³ is OCH₃, R⁴ is H, R⁶ is OCH₃, and R⁷ is OCH₃. In embodiments, R¹ is CH₂OH, R² is H, R³ is CH₂OH, R⁴ is H, R⁶ is OCH₃, and R⁷ is OCH₃. In embodiments, R¹ is OH, R² is H, R³ is CH₂OH, R⁴ is H, R⁶ is OCH₃, and R⁷ is OCH₃. In embodiments, R¹ is OCH₃, R² is H, R³ is CH₂OH, R⁴ is H, R⁶ is OCH₃, and R⁷ is OCH₃. In embodiments, R¹ is OH, R² is H, R³ is OH, R⁴ is H, R⁶ is OH, and R⁷ is OCH₃. In embodiments, R¹ is H, R² is OH, R³ is OH, R⁴ is H, R⁶ is OH, and R⁷ is OCH₃. In embodiments, R¹ is OCH₃, R² is H, R³ is OH, R⁴ is H, R⁶ is OH, and R⁷ is OCH₃. In embodiments, R¹ is OH, R² is H, R³ is OCH₃, R⁴ is H, R⁶ is OH, and R⁷ is OCH₃. In embodiments, R¹ is CH₂OH, R² is H, R³ is OH, R⁴ is H, R⁶ is OH, and R⁷ is OCH₃. In embodiments, R¹ is CH₂OH, R² is H, R³ is OCH₃, R⁴ is H, R⁶ is OH, and R⁷ is OCH₃. In embodiments, R¹ is CH₂OH, R² is H, R³ is CH₂OH, R⁴ is H, R⁶ is OH, and R⁷ is OCH₃. In embodiments, R¹ is OH, R² is H, R³ is CH₂OH, R⁴ is H, R⁶ is OH, and R⁷ is OCH₃. In embodiments, R¹ is OCH₃, R² is H, R³ is CH₂OH, R⁴ is H, R⁶ is OH, and R⁷ is OCH₃. In embodiments, R⁷ can be hydrophobic. In embodiments, R⁷ can be CH₃, CH₂CH₃, 1-propyl, 2-propyl, butyl, or pentyl. In embodiments, R⁷ can be CH₃. In embodiments, R⁷ can be CH₂CH₃. In embodiments, R⁷ can be 1-propyl. In embodiments, R⁷ can be 2-propyl. In embodiments, R⁷ can be butyl. In embodiments, R⁷ can be pentyl.

In general, R⁸ can be any small substituent. Without intending to be bound by theory, it is believed that the presence of certain small substituents on the phenyl ring of the compound according to Formula (I) can improve the binding of the compound to binding sites of bitterness receptors, such as TAS2R¹⁴. In embodiments, R⁸ can be hydrophilic. In embodiments, R⁸ can be H, OH, or OCH₃. In embodiments, R⁸ is H. In embodiments, R⁸ is OH. In embodiments, R⁸ is OCH₃. In embodiments, R¹ is OH, R² is H, R³ is OH, R⁴ is H, R⁶ is OCH₃, R⁷ is OH, and R⁸ is H. In embodiments, R¹ is H, R² is OH, R³ is OH, R⁴ is H, R⁶ is OCH₃, R⁷ is OH, and R⁸ is H. In embodiments, R¹ is OCH₃, R² is H, R³ is OH, R⁴ is H, R⁶ is OCH₃, R⁷ is OH, and R⁸ is H. In embodiments, R¹ is OH, R² is H, R³ is OCH₃, R⁴ is H, R⁶ is OCH₃, R⁷ is OH, and R⁸ is H. In embodiments, R¹ is CH₂OH, R² is H, R³ is OH, R⁴ is H, R⁶ is OCH₃, R⁷ is OH, and R⁸ is H. In embodiments, R¹ is CH₂OH, R² is H, R³ is OCH₃, R⁴ is H, R⁶ is OCH₃, R⁷ is OH, and R⁸ is H. In embodiments, R¹ is CH₂OH, R² is H, R³ is CH₂OH, R⁴ is H, R⁶ is OCH₃, R⁷ is OH, and R⁸ is H. In embodiments, R¹ is OH, R² is H, R³ is CH₂OH, R⁴ is H, R⁶ is OCH₃, R⁷ is OH, and R⁸ is H. In embodiments, R¹ is OCH₃, R² is H, R³ is CH₂OH, R⁴ is H, R⁶ is OCH₃, R⁷ is OH, and R⁸ is H. In embodiments, R¹ is OH, R² is H, R³ is OH, R⁴ is H, R⁶ is OH, R⁷ is OH, and R⁸ is H. In embodiments, R¹ is H, R² is OH, R³ is OH, R⁴ is H, R⁶ is OH, R⁷ is OH, and R⁸ is H. In embodiments, R¹ is OCH₃, R² is H, R³ is OH, R⁴ is H, R⁶ is OH, R⁷ is OH, and R⁸ is H. In embodiments, R¹ is OH, R² is H, R³ is OCH₃, R⁴ is H, R⁶ is OH, R⁷ is OH, and R⁸ is H. In embodiments, R¹ is CH₂OH, R² is H, R³ is OH, R⁴ is H, R⁶ is OH, R⁷ is OH, and R⁸ is H. In embodiments, R¹ is CH₂OH, R² is H, R³ is OCH₃, R⁴ is H, R⁶ is OH, R⁷ is OH, and R⁸ is H. In embodiments, R¹ is CH₂OH, R² is H, R³ is CH₂OH, R⁴ is H, R⁶ is OH, R⁷ is OH, and R⁸ is H. In embodiments, R¹ is OH, R² is H, R³ is CH₂OH, R⁴ is H, R⁶ is OH, R⁷ is OH, and R⁸ is H. In embodiments, R¹ is OCH₃, R² is H, R³ is CH₂OH, R⁴ is H, R⁶ is OH, R⁷ is OH, and R⁸ is H. In embodiments, R¹ is H, R² is OH, R³ is OH, R⁴ is H, R⁶ is OCH₃, R⁷ is OCH₃, and R⁸ is H. In embodiments, R¹ is OCH₃, R² is H, R³ is OH, R⁴ is H, R⁶ is OCH₃, R⁷ is OCH₃, and R⁸ is H. In embodiments, R¹ is OH, R² is H, R³ is OCH₃, R⁴ is H, R⁶ is OCH₃, R⁷ is OCH₃, and R⁸ is H. In embodiments, R¹ is CH₂OH, R² is H, R³ is OH, R⁴ is H, R⁶ is OCH₃, R⁷ is OCH₃, and R⁸ is H. In embodiments, R¹ is CH₂OH, R² is H, R³ is OCH₃, R⁴ is H, R⁶ is OCH₃, R⁷ is OCH₃, and R⁸ is H. In embodiments, R¹ is CH₂OH, R² is H, R³ is CH₂OH, R⁴ is H, R⁶ is OCH₃, R⁷ is OCH₃, and R⁸ is H. In embodiments, R¹ is OH, R² is H, R³ is CH₂OH, R⁴ is H, R⁶ is OCH₃, R⁷ is OCH₃, and R⁸ is H. In embodiments, R¹ is OCH₃, R² is H, R³ is CH₂OH, R⁴ is H, R⁶ is OCH₃, R⁷ is OCH₃, and R⁸ is H. In embodiments, R¹ is OH, R² is H, R³ is OH, R⁴ is H, R⁶ is OH, R⁷ is OCH₃, and R⁸ is H. In embodiments, R¹ is H, R² is OH, R³ is OH, R⁴ is H, R⁶ is OH, R⁷ is OCH₃, and R⁸ is H. In embodiments, R¹ is OCH₃, R² is H, R³ is OH, R⁴ is H, R⁶ is OH, R⁷ is OCH₃, and R⁸ is H. In embodiments, R¹ is OH, R² is H, R³ is OCH₃, R⁴ is H, R⁶ is OH, R⁷ is OCH₃, and R⁸ is H. In embodiments, R¹ is CH₂OH, R² is H, R³ is OH, R⁴ is H, R⁶ is OH, R⁷ is OCH₃, and R⁸ is H. In embodiments, R¹ is CH₂OH, R² is H, R³ is OCH₃, R⁴ is H, R⁶ is OH, R⁷ is OCH₃, and R⁸ is H. In embodiments, R¹ is CH₂OH, R² is H, R³ is CH₂OH, R⁴ is H, R⁶ is OH, R⁷ is OCH₃, and R⁸ is H. In embodiments, R¹ is OH, R² is H, R³ is CH₂OH, R⁴ is H, R⁶ is OH, R⁷ is OCH₃, and R⁸ is H. In embodiments, R¹ is OCH₃, R² is H, R³ is CH₂OH, R⁴ is H, R⁶ is OH, R⁷ is OCH₃, and R⁸ is H. In embodiments, R⁸ can be hydrophobic. In embodiments, R⁸ can be CH₃, CH₂CH₃, 1-propyl, 2-propyl, butyl, or pentyl. In embodiments, R⁸ can be CH₃. In embodiments, R⁸ can be CH₂CH₃. In embodiments, R⁸ can be 1-propyl. In embodiments, R⁸ can be 2-propyl. In embodiments, R⁸ can be butyl. In embodiments, R⁸ can be pentyl.

In general, R⁰ can be any small substituent. Without intending to be bound by theory, it is believed that the presence of certain small substituents on the phenyl ring of the compound according to Formula (I) can improve the binding of the compound to binding sites of bitterness receptors, such as TAS2R14. In embodiments, R⁹ can be hydrophilic. In embodiments, R⁹ can be H, OH, or OCH₃. In embodiments, R⁹ is H. In embodiments, R⁹ is OH. In embodiments, R⁹ is OCH₃. In embodiments, R⁹ can be hydrophobic. In embodiments, R⁹ can be CH₃, CH₂CH₃, 1-propyl, 2-propyl, butyl, or pentyl. In embodiments, R⁹ can be CH₃. In embodiments, R⁹ can be CH₂CH₃. In embodiments, R⁹ can be 1-propyl. In embodiments, R⁹ can be 2-propyl. In embodiments, R⁹ can be butyl. In embodiments, R⁹ can be pentyl.

Also contemplated as a bitter-masking compound is a salt of a compound according to Formula (I) in which one or more hydroxyl groups are deprotonated and one or more cations is present. In these embodiments, the counterion or counterions can include, but are not limited to, potassium, sodium, calcium, ammonium, magnesium, and combinations thereof.

In embodiments, the compound of Formula (I) is 3-(4-hydroxy-3-methoxyphenyl) propane-1,2-diol. In embodiments, the compound of Formula (I) is (2R)-3-(4-hydroxy-3-methoxyphenyl) propane-1,2-diol (hereafter, ‘Compound 1’). Compounds of Formula (I) can exist as racemic mixtures of optical isomers, non-racemic mixtures of optical isomers, or optically pure compounds. Compounds of Formula (I) can be used in pure form or partly purified form and can be synthesized via chemical reactions or via biotechnological procedures including fermentation.

In embodiments, the compound of Formula (I) has the following structure:

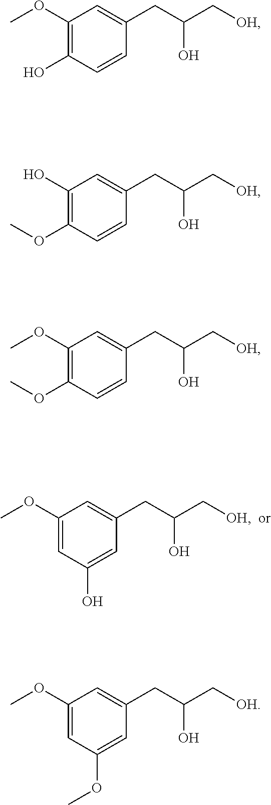

In embodiments, the compound of Formula (I) has the following structure:

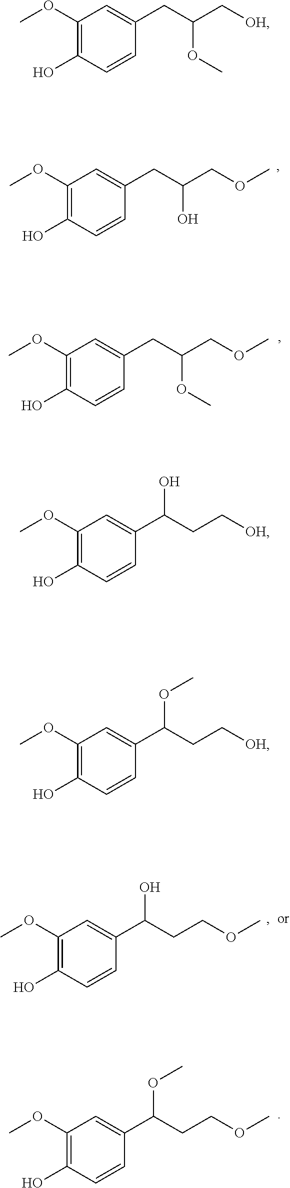

In embodiments, the compound of Formula (I) has the following structure:

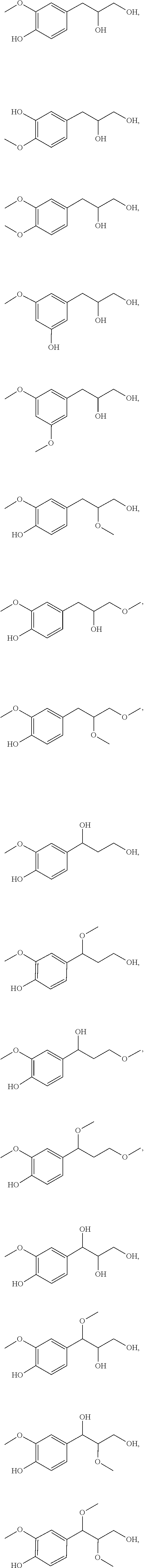

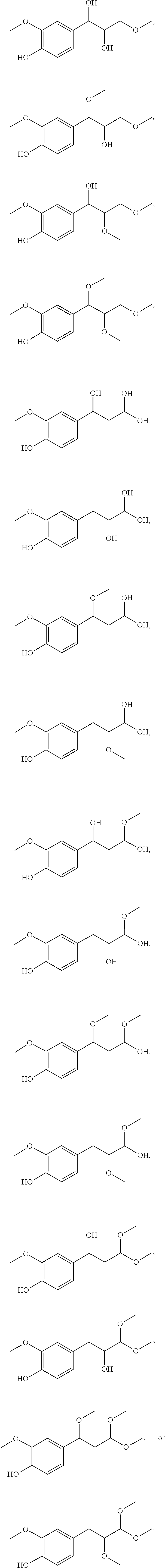

Compounds according to Formula (I) can also be prepared with nutraceuticals using nanocarriers. Suitable nutraceuticals can include, but are not limited to, ginseng, glucosamine, Echinacea, and omega-3 fatty acids. Suitable nanocarriers can include, but are not limited to, carbon nanotubes, nanoemulsions, magnetic nanoparticles, biopolymer matrices, nanoliposomes, solid liquid nanoparticles, mesoporous silica nanoparticles, and dendrimers.

### Ingestible Compositions

Ingestible compositions as described herein can include, but are not limited to, food items, drink items, oral medicaments, or other products placed in the oral cavity. Food and drink items can include, but are not limited to, solid dry consumables (for example, cookies, chocolate, snack, crackers, muffins, energy bars, hard candy, chewing gum, cakes, pastries, biscuits, marshmallow, botanical materials, cereals), dairy products (for example, milk, dairy ice cream, yogurt, milkshake, sour cream, soy milk, dried milk powder, whey, kefir, lassi, frozen yogurt, cheese), drinks (for example, tea, beer, wine, water, mineral water, lemonades, isotonic drinks, vegetable juices, black tea, oolong tea, herbal tea, fruit drink, soft drink, green tea, cacao-based drink, instant coffee), dietary supplements, oil- and fat-based products or emulsions (for example, mayonnaise, dressing), seasonings, and flavorings. Oral medicaments can include, but are not limited to, pharmaceutical or medicinal formulas, in the form of tablets, capsules, granules, pellets, solid mixtures, liquid phase formulas, emulsions, or powders.

Bitter taste in an ingestible composition can arise from the presence of one or more bitter substances in the composition. As used herein, the term “bitter substance” includes, but is not limited to, polyphenols, alkaloids, limonoids, and medicaments, and can include, for instance, flavonoids, tannins, lignans, quinine, caffeine, theobromine, limonin, linoleic acid, denatonium benzoate, amarogentin, fluoroquinolones, aspirin, paracetamol, guaifenesin, ambroxol, salicin, and antibiotics. In embodiments, the bitter substance comprises an alkaloid. In embodiments, the bitter substance comprises quinine. In embodiments, the bitter substance activates TAS2R receptors expressed in oral tissues. In embodiments, the bitter substance activates a TAS2R receptor selected from the group of TAS2R14, TAS2R10, TAS2R46, and a combination thereof. In embodiments, the bitter substance activates the TAS2R14 receptor.

Compounds of Formula (I) can be added to an ingestible composition (such as a food or drink item, an oral medicament, or other product placed in the oral cavity) in an amount sufficient to reduce the perceived bitterness of the composition. In some embodiments, reducing the perceived bitterness of a composition can include reducing the bitterness rating of the composition as evaluated by a trained panel, as described in the Examples section, by at least about 5%, or at least 10%, or at least 15% or at least 20%. Similarly, changing the perceived sweetness of a composition can include changing the sweetness score of the composition as evaluated by a trained panel, as described in the Examples section, by at least about 5%, or at least 10%, or at least 15% or at least 20%. The compounds of Formula (I) and the ingestible composition can be in the form of a solid (e.g., a mixture of a solid compound of Formula (I) and a solid ingestible composition), a liquid mixture or solution (e.g., both the compound of Formula (I) and the ingestible composition are in a liquid state), or a mixture thereof (e.g., a suspension or slurry wherein one of the compound of Formula (I) and the ingestible composition is a solid and the other is a liquid). The compound of Formula (I) and the ingestible composition can optionally be physically separated (e.g., one of the compounds of Formula (I) and/or the ingestible composition can be encapsulated and thus separated from the other by a capsule shell). In embodiments wherein the compound of Formula (I) is provided as a liquid, the compound of Formula (I) can be dissolved in any suitable solvent generally recognized as safe for consumption, including, but not limited to water, ethanol, and a combination thereof.

It will be appreciated that the amount of compound of Formula (I) sufficient to reduce the perceived bitter taste of the ingestible composition will depend on a number of factors, such as, the identity of the bitter substance in the ingestible composition, the extent of perceived bitterness of the bitter substance, the concentration of the bitter substance in the ingestible composition, and the delivery form of the ingestible composition and/or the compound of Formula (I). In general, the higher the intrinsic bitterness of a bitter substance, the more compound of Formula (I) may be needed to reduce the perceived bitterness. Further, as the amount of ingestible composition provided increases, the amount of compound of Formula (I) sufficient to reduce the perceived bitterness may need to increase in order for the compound of Formula (I) to out compete the bitter substance at the taste receptors in the oral cavity. On the other hand, if the compound of Formula (I) is provided in a state that will easily disperse in the oral cavity (e.g., as a liquid), the amount sufficient may depend on the ease of dispersibility of the bitter substance (e.g., less bitter-masking compound may be required where the bitter-masking compound easily disperses as a liquid and the ingestible composition disperses less easily, e.g., as a solid).

In embodiments, the amount of bitter-masking compound sufficient to reduce the perceived bitter taste of an ingestible composition can be in an amount in a range of about 0.001 ppm to about 1000 ppm, based on the amount of the ingestible composition. In embodiments, the amount sufficient can be in a range of about 0.001 ppm to about 500 ppm, about 0.001 ppm to about 250 ppm, about 0.001 ppm to about 100 ppm, about 0.001 ppm to about 90 ppm, about 0.001 ppm to about 80 ppm, about 0.001 ppm to about 75 ppm, about 0.001 ppm to about 70 ppm, about 0.001 ppm to about 60 ppm, about 0.001 ppm to about 50 ppm, about 0.001 ppm to about 40 ppm, about 0.001 ppm to about 30 ppm, about 0.001 ppm to about 25 ppm, about 0.001 ppm to about 20 ppm, about 0.001 ppm to about 15 ppm, about 0.005 ppm to about 15 ppm, about 0.01 ppm to about 15 ppm, about 0.05 ppm to about 15 ppm, about 0.1 ppm to about 15 ppm, about 0.25 ppm to about 15 ppm, about 0.5 ppm to about 15 ppm, about 0.75 ppm to about 15 ppm, about 1 ppm to about 12 ppm, about 2 ppm to about 10 ppm, about 4 ppm to about 8 ppm, about 0.05 ppm to about 1000 ppm, about 0.1 ppm to about 1000 ppm, about 0.5 ppm to about 1000 ppm, about 1 ppm to about 1000 ppm, about 10 ppm to about 1000 ppm, about 50 ppm to about 1000 ppm, about 100 ppm to about 1000 ppm, about 250 ppm to about 1000 ppm, about 500 ppm to about 1000 ppm, about 600 ppm to about 900 ppm, or about 700 ppm to about 800 ppm, based on the amount of the ingestible composition.

The compound of Formula (I) and the ingestible composition can generally be added at any point in time prior to ingestion of the composition, depending on the need. For example, the compound of Formula (I) may be added to the ingestible composition at the point of manufacture and sold ready-to-consume, to enable precise control over the amount of ingestible composition and compound of Formula (I) (e.g., a pharmaceutical prepared with the compound of Formula (I)). As an alternative example, the compound of Formula (I) may be added to the ingestible composition at the time of consumption, to taste. For example, the compound of Formula (I) may be added by a consumer to a prepared ingestible composition, such as a cup of coffee, when the composition is being consumed. In such an embodiment, the compound of Formula (I) can be provided, e.g., as granules or a tincture, to be added to the ingestible composition, to taste.

In embodiments, a flavor profile of an ingestible composition is not changed by more than ±5% upon addition of a compound of Formula (I). In certain embodiments, the level of perceived sweetness of an ingestible composition is not changed by more than ±5% by addition of a compound of Formula (I). For instance, as shown in Example 4 below, the perceived sweetness of a sucrose solution was changed by no more than ±5% upon addition of various amounts of a compound of Formula (I). Thus, in some embodiments, the flavor profile is not changed by more than ±5% upon addition of a compound of Formula (I), while in other embodiments, for example, in the presence of a bitter substance and a sweetener, the flavor profile may have a perceived increase in sweetness due in part to attenuation of perceived bitterness.

Compounds according to Formula (I) can be added to an ingestible composition in combination with one or more other agents that can affect the taste of the composition. For instance, compounds according to Formula (I) can be combined with other agents that further mask or inhibit bitterness, including but not limited to cyclodextrins, sodium caseinate, oleic acid, homoeriodictyol, eriodictyol, sterubin, 6-methoxyflavanone, sodium ferulate salt, homoeriodictyol disodium salt, eriodictyol 7-methyl ether, N-(1-carboxyethyl)-3-hydroxy-6-hydroxymethyl pyridinium inner salt, N-carboxymethyl-3-hydroxy-6-hydroxymethyl-pyridinium inner salt, N-(1,3-dicarboxypropyl)-3-hydroxy-6-hydroxymethyl-pyridinium inner salt, (S)—N-(1-carboxyethyl)-3-hydroxy-6-hydroxy-methylpyridinium inner salt, (R)—N-(1-carboxyethyl)-3-hydroxy-6-hydroxy-methyl-pyridinium inner salt, (S)—N-[N-(1-carboxy-2-phenylethyl)-carboxamido-methyl]-3-hydroxy-6-hydroxymethyl-pyridinium inner salt, N-[1-[(3,5-dimethyl-4-isoxazolyl)methyl]-1H-pyrazol-4-yl]-α-methylbenzeneacetamide, N-[1-[(3,5-dimethyl-4-isoxazolyl)methyl]-1H-pyrazol-4-yl]-3-methoxybenzamide, and 4-[[(4-methoxyphenyl)methyl](phenylmethyl)amino]sulfonyl]benzoic acid.

Compounds according to Formula (I) can also be combined with one or more flavor-enhancing agents to enhance the flavor of an ingestible composition. Flavor-enhancing agents can include but are not limited to menthol, almond, vanillin, vanilla extract, cinnamon, citrus extract, ginger, grape extract, and coconut.

Compounds according to Formula (I) can also be combined with one or more sweetening agents to enhance the sweetness of an ingestible composition. Sweetening agents can include but are not limited to cyclamate, neotame, aspartame, saccharin, neohesperidin dihydrochalcone, advantame, acesulfame, sucralose, thaumatin, neoculin, miraculin, brazzein, mabinlin-2, stevioside, stevia, rubososide, Luo han guo extract, mogroside V, mogroside VI, abrusoside A, dulcoside A-B, rebaudiosides A-F, rebaudiosides H-L, rebaudiosides N—O, glycyrrhizic acid, telosmoside A15, siamenoside I, pediandrin I-V, albiziasaponin A, baiyunoside, millettiasaponin A, apioglycyrrhizin, araboglycyrrhizin, derrisaponin A, hernandulcin, perillatine, sucrose, glucose, fructose, rhamnose, xylose, arabinose, ribose, ribulose, allose, galactose, mannose, monellin, hesperetin, FEMA GRAS enhancer 4601, FEMA GRAS enhancer 4728, FEMA GRAS enhancer 4774, FEMA GRAS enhancer 4728, FEMA GRAS enhancer 4701, FEMA GRAS enhancer 4720, FEMA GRAS enhancer 4469, FEMA GRAS enhancer 4708, 4-amino benzoic acid, 3-amino benzoic acid, 2,3,4-trihydroxybenzoic acid, 2,6-dihydrobenzoic acid, 2,4,6-trihydroxybenzoic acid, 2,4-dihydroxybenzoic acid, 4-hydrobenzoic acid, 3-hydroxybenzoic acid, and 2-hydroxybenzoic acid.

The disclosure further provides a method of obtaining (2R)-3-(4-hydroxy-3-methoxyphenyl) propane-1,2-diol from allspice. The method generally includes admixing allspice with an extraction solvent to form a crude mixture, collecting a liquid phase of the crude mixture, and collecting (2R)-3-(4-hydroxy-3-methoxyphenyl) propane-1,2-diol from the liquid phase.

The method of obtaining (2R)-3-(4-hydroxy-3-methoxyphenyl) propane-1,2-diol from allspice comprises:
(a) admixing allspice with an extraction solvent to form a crude mixture having a liquid phase and a solid phase, wherein the extraction solvent comprises a mixture of water and ethanol, containing from 70 wt. % to 90 wt. % ethanol, based on the total weight of the solvent; and
(b) collecting a first portion of the liquid phase, wherein the first portion of the liquid phase would elute from a liquid chromatography column using a first elution solution comprising water and ethanol containing about 20 wt. % to about 40 wt. % ethanol based on the total weight of the first elution solution,
wherein the first portion of the liquid phase comprises (2R)-3-(4-hydroxy-3-methoxyphenyl) propane-1,2-diol.

While the method of obtaining (2R)-3-(4-hydroxy-3-methoxyphenyl) propane-1,2-diol from allspice can employ solvents in addition to water and ethanol, the method is preferably carried out using water and ethanol as the only solvents, in order to minimize or eliminate contamination of (2R)-3-(4-hydroxy-3-methoxyphenyl) propane-1,2-diol so obtained with materials that are not generally recognized as safe for human consumption.

Admixing allspice with an extraction solvent comprising a mixture of water and ethanol provides a crude mixture comprising a liquid phase and a solid phase, wherein the liquid phase can contain (2R)-3-(4-hydroxy-3-methoxyphenyl) propane-1,2-diol. The extraction solvent can comprise 100 wt. % water, or 100 wt. % ethanol, or any mixture of ethanol and water. For example, the extraction solvent can comprise a mixture of water and ethanol containing ethanol in an amount of at least about 1 wt. %, 5 wt. %, 10 wt. %, 20 wt. %, 30 wt. %, 40 wt. %, 50 wt. %, 60 wt. %, or 70 wt. %, and at most about 99 wt. %, 95 wt. %, 90 wt. %, or 80 wt. %, based on the total weight of the extraction solvent, for example about 1 wt. %, 10 wt. %, 20 wt. %, 30 wt. %, 40 wt. %, 50 wt. %, 60 wt. %, 70 wt. %, 80 wt. %, or 90 wt. % or in a range of about 30 wt. % to about 95 wt. %, about 40 wt. % to about 95 wt. %, about 50 wt. % to about 90 wt. %, about 60 wt. % to about 90 wt. %, or about 70 wt. % to about 90 wt. %. Without intending to be bound by theory, it is believed that using an extraction solvent comprising a mixture of water and ethanol can be more effective at extracting compounds having a broader range of solubilities compared to using an extraction solvent comprising neat water or neat ethanol. It is further believed that mixtures comprising water and ethanol comprising 70 wt. % to 90 wt. % ethanol can more effectively extract higher molecular weight compounds, including compounds having aromatic groups, from allspice compared to mixtures comprising water and ethanol having lower ethanol content.

The admixing of allspice with the extraction solvent can be performed at a temperature in a range of about 20° C. to about 80° C., for example, about 20° C. to about 75° C., or about 25° C. to about 75° C., or about 30° C. to 75° C., or about 30° C. to about 70° C., or about 35° C. to about 70° C., from about 35° C. to about 65° C., or about 40° C. to about 65° C., or about 40° C. to about 60° C., or about 45° C. to about 60° C., or about 45° C. to about 55° C.

The method of obtaining (2R)-3-(4-hydroxy-3-methoxyphenyl) propane-1,2-diol from allspice includes collecting a first portion of the liquid phase that would elute from a chromatography column using a first elution solution (i.e., mobile phase) comprising water and ethanol containing about 20 wt. % to about 40 wt. % ethanol based on the total weight of the first elution solution, wherein the first portion of the liquid phase comprises (2R)-3-(4-hydroxy-3-methoxyphenyl) propane-1,2-diol. The first portion of the liquid phase can be collected by any suitable means. In embodiments, the first portion of the liquid phase is collected by loading the liquid phase onto a chromatography column and eluting the liquid phase from the column using a first elution solution comprising water and ethanol containing about 20 wt. % to about 40 wt. % ethanol based on the total weight of the first elution solution. The liquid phase can optionally be concentrated, for instance via solvent removal, prior to being loaded onto the chromatography column.

In embodiments, the method of obtaining (2R)-3-(4-hydroxy-3-methoxyphenyl) propane-1,2-diol from allspice also includes collecting a second portion of the liquid phase that would elute from a chromatography column using a second elution solution (i.e., mobile phase) comprising water and ethanol containing about 40 wt. % to about 60 wt. % ethanol based on the total weight of the second elution solution, wherein the second portion of the liquid phase comprises (2R)-3-(4-hydroxy-3-methoxyphenyl) propane-1,2-diol. The second portion of the liquid phase can be collected by any suitable means. In embodiments, the second portion of the liquid phase is collected by loading the liquid phase onto a chromatography column and eluting the liquid phase from the column using a second elution solution comprising water and ethanol containing about 40 wt. % to about 60 wt. % ethanol based on the total weight of the second elution solution. The liquid phase can optionally be concentrated, for instance via solvent removal, prior to being loaded onto the chromatography column.

### Sensory Testing

Sensory testing was used to identify extracts that provide bitterness reduction and to evaluate the degree of bitterness reduction provided by specific compounds. In general, bitterness sensory testing involved providing test solutions to trained panelists who rated the perceived bitterness of test solutions. Panels consisted of three persons who can taste phenylthiocarbamide (PTC) test paper, and panelists were selected based on their ability to discriminate differences in the concentrations of bitterness. Panelists swirled an aliquot of a solution in the mouth for 15 seconds, spit out the test solution, and rated the perceived bitterness intensity of the solution on a scale of 0 to 15 (0: no bitterness, 15: very bitter). Panelists drank water after each test solution evaluation to remove the bitterness before evaluating the next solution.

Similar panel testing was done to evaluate perceived sweetness of test solutions. Panelists swirled an aliquot of test solution in the mouth for 15 seconds, spit out the test solution, and rated the perceived sweetness intensity of the test solution on a scale of 0 to 15 (0: no sweetness, 15: very sweet). Panelists drank water after each test solution evaluation before evaluating the next test solution.

Panel testing results can be used to quantify perceived changes in bitterness. The change in perceived bitterness of an ingestible composition following addition of a bitter-masking agent is defined herein as the percent change in the bitterness panel test rating of a composition to which a bitter-masking agent has been added compared to the bitterness panel test rating of the otherwise identical composition without the bitter-masking agent. For instance, if a composition is given a bitterness intensity rating of 8, and the same composition with addition of an amount of bitter-masking agent is given a bitterness intensity rating of 6, then the bitter-masking agent has provided a perceived change in bitterness of 25%.

Panel testing results can be used to quantify perceived changes in sweetness in an analogous manner. The change in perceived sweetness of an ingestible composition following addition of a bitter-masking agent or other agent is defined herein as the percent change in the sweetness panel test rating of a composition to which a bitter-masking agent or other agent has been added compared to the sweetness panel test rating of the otherwise identical composition without the bitter-masking agent or other agent. For instance, if a composition is given a sweetness intensity rating of 8, and the same composition with addition of an amount of bitter-masking agent or other agent is given a sweetness intensity rating of 6, then the bitter-masking agent or other agent has provided a perceived change in sweetness of 25%.

### Molecular Docking

Taste perception is mediated by TAS1R or TAS2R receptors that are expressed in the oral tissues. TAS1R genes are composed of 3 isoforms, including TAS1R1, TAS1R2, and TAS1R3, that are receptors for sweet and umami taste; TAS2R genes consist of 25 isoforms that are bitter taste receptors. Of the 25 bitter taste receptors, TAS2R14, TAS2R10 and TAS2R46 are among the most highly tuned; the combined response of these three recognizes approximately 50% of bitterness, and each accounts for one-third of total bitterness. TAS2R14 is known to be activated by a wide range of stimuli, including natural products and synthetic substances. Accordingly, finding an antagonist to TAS2R14 is critical to elucidate the mechanism of bitter taste inhibition. Molecular docking analysis, using molecular models of the structure of the binding pocket of a receptor and the structure of a potential antagonist, can be used to evaluate how strongly the potential antagonist is expected to bind to the receptor and thus how effectively the potential antagonist is expected to be at reducing perceived bitter taste.

Molecular docking analysis was performed on GOLD software version 5.3 (Genetic Optimisation for Ligand Docking; the Cambridge Crystallographic Data Centre, Cambridge, UK) with default parameters. The structure of bitter receptor TAS2R14 was downloaded from the BitterDB Protein Data Bank (http://bitterdb.agri.huji.ac.il/dbbitter.php) (BitterDB ID: 14). The binding pocket for GOLD molecular docking calculations was defined based on previous literature and PrankWeb prediction (https://prankweb.cz/). The best GOLD PLP CHEM score was chosen and Visualization of 2D models of complexes was depicted by BIOVIA Discovery Studio 2021.

As described in the Examples, below, Compound 1 was docked into the binding site of TAS2R14 using the GOLD 3.01 docking program. Asn 93 formed a hydrogen bond with two oxygens attached to the aryl ring of Compound 1. The methoxy group on the aryl ring also interacted with Phe 186 and Trp 89 through TT-alkyl hydrophobic bonds. The aryl ring also formed TT-TT interactions with Phe 247 and Trp 89. Accordingly, due to the structural similarity between the compounds having a structure according to Formula 1 and Compound 1, the compounds having a structure according to Formula 1 are expected to function in the same way as Compound 1 with respect to inhibiting perceived bitter taste through the TAS2R14 receptor. In particular, compounds having a structure according to Formula (I), having the same structural backbone as Compound 1, an aryl ring as in Compound 1, and oxygen atoms attached to the aryl ring as in Compound 1, are expected to inhibit perceived bitter taste of an ingestible composition. Without intending to be bound by theory, the presence of multiple interactions between Compound 1 and the binding pocket of TAS2R14 as indicated by molecular docking analysis suggests that Compound 1 is expected to bind strongly to TAS2R14 and inhibit perceived bitter taste by acting as an antagonist of TAS2R14.

### EXAMPLES

### Example 1: Extraction and Isolation of Bitter-Masking Compounds

Extracts from 36 spices were evaluated by sensory testing to assess which spices contain compounds that are effective at masking bitterness. Spices were treated with boiling water, the resulting crude liquid phases were concentrated via rotary evaporation, and the resulting residues were freeze-dried to obtain crude extracts. Crude extracts were evaluated for bitter-masking activity by panel testing. For each crude extract, the panel compared the perceived bitterness of a control solution containing 100 ppm quinine in distilled water to that of a test solution containing 100 ppm quinine and a sample of the crude extract. None of the crude extracts was found to increase the perceived sweetness of a 4% sucrose solution, as determined by panel testing. Eight spices whose crude extracts reduced the perceived bitterness of the quinine control solution were selected for further evaluation.

Spices were extracted using twice with 30% ethanol at 45° C. and twice with 80% ethanol at 45° C. The liquid phases from each extraction were combined and concentrated by rotary evaporation, and the resulting concentrated extract loaded onto a SP-70 column and eluted with water, aqueous ethanol solutions containing 30%, 50%, and 70% ethanol, and ethanol. An elution fraction was collected for each mobile phase, each elution fraction was concentrated via rotary evaporation to yield a residue, and each residue was freeze-dried. The dried residues were subjected to sensory evaluation for bitterness reduction against a quinine control solution. Of these samples, the residue from the 30% ethanol and 50% ethanol fractions of the extract from allspice showed the most potent bitter-masking activity. Allspice was thus selected for further analysis.

Allspice was extracted three times with 80% ethanol at room temperature using sonication. The combined liquid phases were dried via rotary evaporation. The resulting residue was loaded onto an SP-70 column and eluted using a step-gradient mobile phase system of water, ethanol:water solutions containing 30%, 50%, and 70% ethanol, and ethanol. An elution fraction was collected for each mobile phase, each elution fraction was concentrated via rotary evaporation to yield a residue, and each residue was dried and analyzed via sensory testing. The 30% ethanol elution fraction showed the most potent bitter-reducing activity, and the 50% ethanol elution fraction showed moderate bitter-reducing activity.

The 30% ethanol elution fraction was separated via flash chromatography to obtain four sub-fractions, the second and third of which showed significant bitter-reducing activity in sensory testing. The third sub-fraction was separated using a Sephadex LH-20 column using a gradient mobile phase system (70:30 ethanol:water to 100 ethanol) to obtain two further sub-fractions; preparative HPLC using a reversed-phase HPLC column was used to isolate (2R)-3-(4-hydroxy-3-methoxyphenyl) propane-1,2-diol (Compound 1) from the second fraction. Compound 1 was also isolated from the 50% ethanol elution fraction. LC-MS quantification analysis showed that the amount of Compound 1 in allspice was 1.248±0.071 ppm.

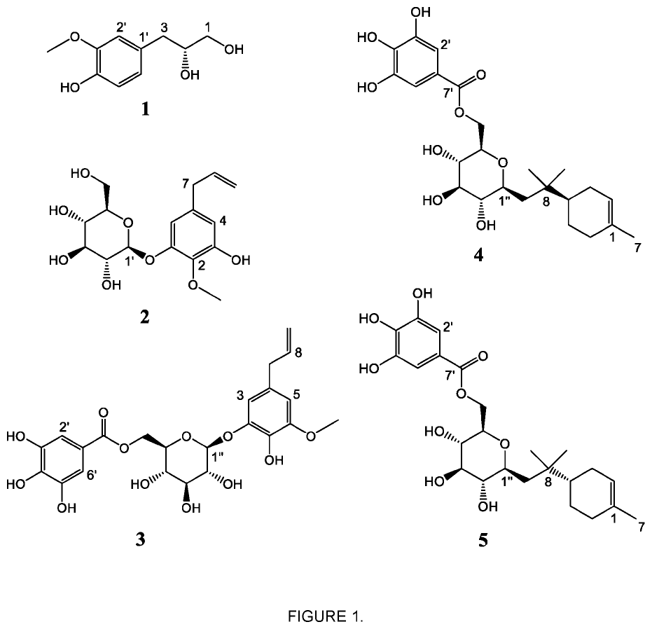

Compounds 2-5 were also isolated from allspice extracts. The structures of Compounds 1-5, including numerical identification of carbon atoms referenced in the spectroscopic characterizations described below, are shown in FIG. 1.

Spectroscopic data for Compound 1: ¹H-NMR (methanol-d₄; 600 MHz): 3.50 (1H, dd, J=11.2, 4.4 Hz, H-1a), 3.43 (1H, dd, J=11.2, 6.2 Hz, H-1b), 3.77 (1H, m, H-2), 2.73 (1H, dd, J=13.8, 5.8 Hz, H-3a), 2.60 (1H, dd, J=13.8, 7.4 Hz, H-3b), 6.82 (1H, d, J=1.9 Hz, H-2′), 6.71 (1H, d, J=8.0 Hz, H-5′), 6.66 (1H, dd, J=8.0, 1.9 Hz, H-6′), 3.84 (3H, s, OMe). ¹³C-NMR (methanol-d₄; 150 MHz): 66.5 (C-1), 74.7 (C-2), 40.5 (C-3), 131.6 (C-1′), 114.1 (C-2′), 148.8 (C-3′), 145.9 (C-4′), 116.0 (C-5′), 122.9 (C-6′), 56.3 (OMe).

### Structural Identification of Compound 1:

Compound 1 was isolated as a colorless oil. High-resolution electrospray ionization mass spectrometry (HRESIMS) data (m/z 197.0818 [M-H], vs. calcd. m/z 197.0813 for C₁₀H₁₃O₄) suggested a molecular formula of C₁₀H₁₄O₄. Mass spectrometry and NMR data established the chemical structure of Compound 1. Three aromatic protons were shown at [6.82 (1H, d, J=1.9 Hz, H-2′), 6.71 (1H, d, J=8.0 Hz, H-5′), 6.66 (1H, dd, J=8.0, 1.9 Hz, H-6′)], which suggested typical ABX system. One pair of ortho-positioned and one pair of meta-positioned protons were observed in this ABX aromatic system. One pair of ortho positioned protons were presented at [6.71 (1H, d, J=8.0 Hz, H-5′), 6.66 (1H, dd, J=8.0, 1.9 Hz, H-6′)] with the coupling constant of 8.0 Hz. Two protons [6.82 (1H, d, J=1.9 Hz, H-2′), 6.66 (1H, dd, J=8.0, 1.9 Hz, H-6′)] showed coupling constant of 1.9 Hz, indicating they are meta protons. Corresponding carbons of aromatic protons were confirmed based on heteronuclear single quantum coherence (HSQC) spectrum (δH 6.82/δC 114.1, δH 6.71/δC 116.0, δH 6.66/δC 122.9). Two out of six carbons on the aryl ring showed relatively deshielded chemical shift at oC 145.9 and 148.8 indicating that they are substituted with oxygen. The heteronuclear multiple bond correlation (HMBC) correlation from methoxy group (δH 3.84, s, 3H) to δC 148.8 confirmed the substitution of methoxy group on benzene ring. Previous literature confirmed the position of methoxy group on benzene ring with the help of HMBC correlations (H-6′/C-2′ and C-4′). One pair of meta-positioned aromatic protons [6.82 (1H, d, J=1.9 Hz, H-2′), 6.66 (1H, dd, J=8.0, 1.9 Hz, H-6′)] showed HMBC correlations with C-3 (δC 40.5). HSQC correlated two methylene protons [2.73, (1H, dd, J=13.8, 5.8 Hz), 2.60 (1H, dd, J=13.8, 7.4 Hz)] to C-3 methylene carbon. These two methylene protons have HMBC correlation with methine (C-2, δC 74.7). Remaining methylene protons (H-1) were correlated with C-2 based on HMBC spectrum. H-1a (δH 3.50, dd, J=11.2, 6.2 Hz, 1H) was geminally coupled with H-1b (δH 3.43, dd, J=11.2, 6.2 Hz, 1H). Carbon chemical shifts of this compound were in a good agreement with previous literature. Therefore, Compound 1 was characterized as (2R)-3-(4-hydroxy-3-methoxy-phenyl) propane-1,2-diol.

Spectroscopic data for Compound 2: ¹H-NMR (methanol-d₄; 600 MHz): 6.55 (1H, d, J=1.8 Hz, H-4), 6.72 (1H, d, J=1.8 Hz, H-6), 3.29 (2H, d, J=6.6 Hz, H-7), 5.97 (1H, tdd, J=17.5, 10.5, 6.5 Hz, H-8), 5.09 (1H, dd, J=17.0, 2.0 Hz, H-9a), 5.04 (1H, dd, J=10.0, 2.0 Hz, H-9b), 4.76 (1H, d, J=7.5 Hz, H-1″), 3.43 (1H, m, H-2″), 3.48 (1H, m, H-3″), 3.41 (1H, m, H-4″), 3.52 (1H, m, H-5″), 3.90 (1H, dd, J=12.0, 1.5 Hz, H-6″a), 3.74 (1H, dd, J=12.0, 5.4 Hz, H-6″b). ¹³C-NMR (methanol-d₄; 150 MHz): 146.9 (C-1), 149.6 (C-2), 135.8 (C-3), 108.5 (C-4), 132.5 (C-5), 111.7 (C-6), 41.0 (C-7), 139.1 (C-8), 115.8 (C-9). HRESIMS peak at m/z 341.1241 [M-H]⁻ (calcd, for C₁₆H₂₁O₈, 341.1236).

Spectroscopic data for Compound 3: ¹H-NMR (methanol-d₄; 600 MHz): 6.55 (1H, d, J=1.8 Hz, H-3), 6.48 (1H, d, J=1.8 Hz, H-5), 3.04 (2H, dd, J=15.6, 6.6 Hz, H-7), 5.78 (1H, m, H-8), 4.92 (1H, m, H-9), 7.10 (2H, s, H-2′, 6′), 4.75 (1H, d, J=7.2 Hz, H-1″), 3.51 (1H, m, H-2″), 3.53 (1H, m, H-3″), 3.51 (1H, m, H-4″), 3.75 (1H, m, H-5″), 4.61 (1H, dd, J=12.0, 2.4 Hz, H-6″a), 4.48 (1H, dd, J=12.0, 6.6 Hz, H-6″b), 3.81 (3H, s, OMe). ¹³C-NMR (methanol-d₄; 150 MHz): 146.8 (C-1), 135.0 (C-2), 149.5 (C-3), 108.5 (C-4), 132.6 (C-5), 111.6 (C-6), 40.7 (C-7), 139.0 (C-8), 115.6 (C-9), 140.0 (C-1′), 110.2 (C-2′, 6′), 146.6 (C-3′, 5′), 121.3 (C-4′), 168.3 (C-7′), 104.2 (C-1″), 75.0 (C-2″), 77.4 (C-3″), 71.8 (C-4″), 76.0 (C-5″), 64.8 (C-6″), 56.7 (OMe). HRESIMS peak at m/z 493.1352 [M-H]⁻ (calcd, for C₂₃H₂₅O₁₂, 493.1346).

Spectroscopic data for Compound 4: ¹H-NMR (methanol-d₄; 600 MHz): 5.29 (1H, m, H-2), 2.01 (1H, m, H-3), 1.62 (1H, m, H-4), 1.97 (1H, m, H-5a), 1.14 (1H, m, H-5b), 1.89 (1H, m, H-6a), 1.81 (1H, m, H-6b), 1.50 (3H, br s, H-7), 1.14 (3H, s, H-9), 1.18 (3H, s, H-10), 7.13 (1H, m, H-2′, 6′), 4.53 (1H, d, J=7.8 Hz, H-1″), 3.18 (1H, t, J=8.4 Hz, H-2″), 3.45 (1H, t, J=8.8 Hz, H-3″), 3.40 (1H, t, J=9.1 Hz, H-4″), 3.60 (1H, t, J=8.5 Hz, H-5″), 4.50 (1H, m, H-6″a), 4.28 (1H, dd, J=11.4, 6.6 Hz, H-6″b). ¹³C-NMR (methanol-d₄; 150 MHz): 134.3 (C-1), 121.8 (C-2), 27.6 (C-3), 44.6 (C-4), 24.6 (C-5), 31.6 (C-6), 23.5 (C-7), 79.9 (C-8), 23.0 (C-9), 25.1 (C-10), 138.6 (C-1′), 109.9 (C-2′, 6′), 146.0 (C-3′, 5′), 121.9 (C-4′), 166.8 (C-7′), 98.2 (C-1″), 75.0 (C-2″), 78.0 (C-3″), 71.7 (C-4″), 74.6 (C-5″), 65.0 (C-6″). HRESIMS peak at m/z 467.1918 [M-H]⁻ (calcd, for C₂₃H₃₁O₁₀, 467.1917).

Spectroscopic data for Compound 5: ¹H-NMR (methanol-d₄; 600 MHz): 5.30 (1H, m, H-2), 2.02 (1H, m, H-3), 1.59 (1H, m, H-4), 1.96 (1H, m, H-5a), 1.17 (1H, m, H-5b), 1.91 (1H, m, H-6a), 1.85 (1H, m, H-6b), 1.56 (3H, br s, H-7), 1.15 (3H, s, H-9), 1.18 (3H, s, H-10), 7.12 (1H, m, H-2′, 6′), 4.53 (1H, m, H-1″), 3.17 (1H, t, J=8.4 Hz, H-2″), 3.43 (1H, t, J=8.4 Hz, H-3″), 3.36 (1H, t, J=9.0 Hz, H-4″), 3.57 (1H, t, J=7.2 Hz, H-5″), 4.51 (1H, m, H-6″a), 4.27 (1H, dd, J=12.0, 7.2 Hz, H-6″b). ¹³C-NMR (methanol-d₄; 150 MHz): 134.2 (C-1), 121.8 (C-2), 27.5 (C-3), 44.6 (C-4), 24.5 (C-5), 31.6 (C-6), 23.6 (C-7), 79.8 (C-8), 24.0 (C-9), 24.2 (C-10), 138.7 (C-1′), 109.9 (C-2′, 6′), 146.0 (C-3′, 5′), 121.9 (C-4′), 166.7 (C-7′), 98.1 (C-1″), 75.0 (C-2″), 78.1 (C-3″), 71.7 (C-4″), 74.6 (C-5″), 64.9 (C-6″). HRESIMS peak at m/z 467.1915 [M-H]⁻ (calcd. for C₂₃H₃₁O₁₀, 467.1917).

### Sensory Testing

### Example 2

Compound 1 was evaluated by sensory panel testing. A solution of 6 ppm quinine monohydrochloride dihydrate in distilled water was used as a control solution having high bitterness, and Compound 1 was added to aliquots of the control solution to provide test solutions containing 6 ppm quinine and 25, 50, or 100 ppm of Compound 1. Results of sensory panel testing are shown in Table 1.

TABLE 1

| | Panel Score |
|---|---|
| Description | (0-15) |
| 6 ppm quinine | 6.0 |
| 6 ppm quinine + 25 ppm Compound 1 | 5.0 |
| 6 ppm quinine + 50 ppm Compound 1 | 3.0 |
| 6 ppm quinine + 100 ppm Compound 1 | 0.8 |

The panel results show that Compound 1 markedly reduced the perceived bitterness of quinine solutions. The continuous decrease in perceived bitterness with increased concentration of Compound 1 demonstrates that Compound 1 was responsible for the bitterness reduction.

Compounds 2-5 were also evaluated by sensory panel testing in the same manner as Compound 1. None of Compounds 2-5 inhibited the bitterness of quinine.

Compound 1 was evaluated by sensory panel testing for bitterness reduction of caffeine solutions. A solution of 500 ppm caffeine in distilled water was used as a control solution having high bitterness, and Compound 1 was added to aliquots of the control solution to provide test solutions containing 500 ppm caffeine and 25, 50, or 100 ppm of Compound 1. Results of sensory panel testing are shown in Table 2.

TABLE 2

| | Panel Score |
|---|---|
| Description | (0-15) |
| 500 ppm caffeine | 3.0 |
| 500 ppm caffeine + 25 ppm Compound 1 | 3.5 |
| 500 ppm caffeine + 50 ppm Compound 1 | 4.0 |
| 500 ppm caffeine + 100 ppm Compound 1 | 3.8 |

Table 2 indicates that Compound 1 did not significantly mask the bitterness of caffeine solutions. Without intending to be bound by theory, it is believed that caffeine interacts with multiple bitterness receptors and that the interactions of caffeine with multiple receptors can outcompetes the bitter reducing effects of Compound 1.

### Example 3

Sensory panel tests were also performed to evaluate whether crude extracts from allspice would be effective at reducing bitterness. Allspice was mixed with boiling water to obtain a crude aqueous mixture, and solvent was removed from the crude aqueous mixture using rotary evaporation to obtain a crude extract. The crude extract was used without further purification. An aqueous solution containing 6 ppm quinine was used as a bitterness control solution, and the crude extract was added to aliquots of the control solution to provide test solutions containing 6 ppm quinine and 100 ppm or 1000 ppm of the crude extract. The three solutions were evaluated by a sensory panel as described above. Results of sensory panel testing are shown in Table 3.

TABLE 3

| | Panel Score |
|---|---|
| Description | (0-15) |
| 6 ppm quinine | 6.0 |
| 6 ppm quinine + 100 ppm crude | 7.4 |
| allspice extract | |
| 6 ppm quinine + 1000 ppm crude | 7.4 |
| allspice extract | |

The sensory panel data show that crude extracts from allspice did not reduce the perceived bitterness of quinine, even though the crude extracts contain compounds (e.g., Compound 1) that do reduce the perceived bitterness of quinine. Without intending to be bound by theory, it is believed that the concentration of Compound 1 in the crude allspice extract was insufficient to provide bitterness masking and/or that allspice may contain compounds that impart bitterness or that inhibit the bitter-masking activity of Compound 1.

### Example 4

A similar panel test was performed to assess whether the efficacy of Compound 1 at masking bitterness is derived from imparting sweetness. A solution of 6 ppm sucrose in distilled water was used as a sweetness control solution, and Compound 1 was added to three aliquots of the sweetness control solution to provide test solutions containing 6 ppm sucrose and 25, 50, or 100 ppm of Compound 1. Panelists then evaluated the sweetness intensity of each test solution. Sensory evaluation results are shown in Table 4.

TABLE 4

| | Panel Score |
|---|---|
| Description | (0-15) |
| 6 ppm sucrose | 7.0 |
| 6 ppm sucrose + 25 ppm Compound 1 | 7.2 |
| 6 ppm sucrose + 50 ppm Compound 1 | 6.8 |
| 6 ppm sucrose + 100 ppm Compound 1 | 7.2 |

The panel test results show that Compound 1 did not contribute to perceived sweetness of the test solutions, indicating that the bitter-masking activity of Compound 1 is not due to imparting sweetness.

### Example 5: Molecular Docking

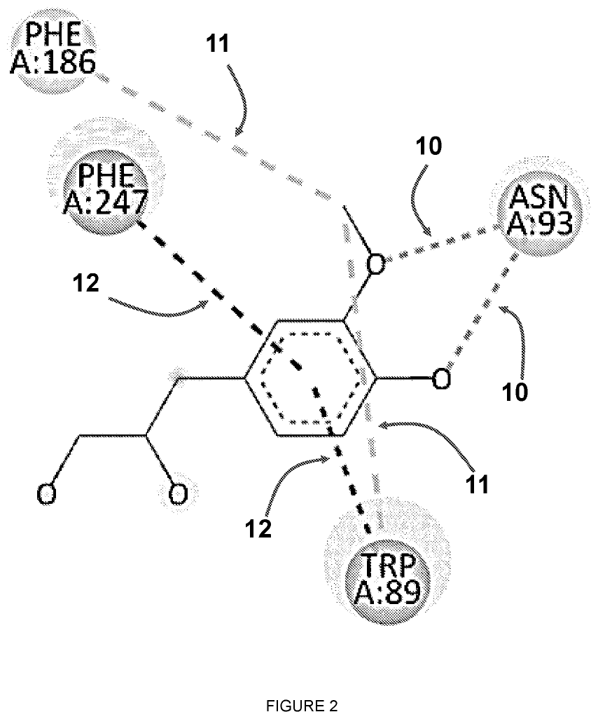

Following the isolation and structural characterization of Compound 1, molecular docking analysis of Compound 1 was performed. Compound 1 was docked into the binding site of TAS2R14 using the GOLD 3.01 docking program. FIG. 2 shows a graphical representation of the results of the molecular docking analysis. Asn 93 formed hydrogen bonds 10 with two oxygens attached to the aryl ring of Compound 1. The methoxy group on the aryl ring also interacted with Phe 186 and Trp 89 through TT-alkyl hydrophobic bonds 11. The aryl ring also formed TT-TT interactions 12 with Phe 247 and Trp 89. Without intending to be bound by theory, the presence of multiple interactions between Compound 1 and the binding pocket of TAS2R14 as indicated by molecular docking analysis suggests that Compound 1 is expected to bind strongly to TAS2R14 and inhibit perceived bitter taste by acting as an antagonist of TAS2R14.

What is claimed is:

1. A method of masking bitter taste of an ingestible composition, comprising adding a bitter-masking compound to the ingestible composition, wherein the bitter-masking compound comprises a compound according to Formula (I) 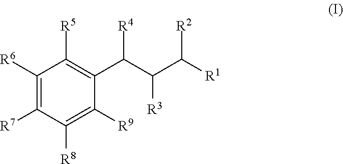
wherein R¹, R², R³, R⁴, R⁵, R⁶, R⁷, R⁸ and R⁹ are independently H, CH₃, CH₂CH₃, 1-propyl, 2-propyl, butyl, pentyl, OH, OCH₃, OCH₂CH₃, 1-propyloxy, 2-propyloxy, C₁₋₃alkylene-OH, C₁₋₃alkylene-OCH₃, acyl, substituted acyl, aryl, or substituted aryl.

2. The method of claim 1, wherein R¹ and R³ are independently H, OH, OCH₃, C₁₋₃alkylene-OH, or C₁₋₃alkylene-OCH₃, R², R⁴, R⁶, R⁷, and R⁸ are independently H, OH, or OCH₃, and R⁵ and R⁹ are H.

3. The method of claim 1, wherein R¹ is OH.

4. The method of claim 1, wherein R¹ is OCH₃.

5. The method of claim 1, wherein R¹ is CH₂OH.

6. The method of any one of claims 1 to 5, wherein R² is H.

7. The method of any one of claims 1 to 5, wherein R² is OH.

8. The method of any one of claims 1 to 7, wherein R³ is OH.

9. The method of any one of claims 1 to 7, wherein R³ is OCH₃.

10. The method of any one of claims 1 to 7, wherein R³ is CH₂OH.

11. The method of any one of claims 1 to 10, wherein R⁴ is H.

12. The method of any one of claims 1 to 10, wherein R⁴ is OH.

13. The method of any one of claims 1 to 12, wherein R⁶ is H.

14. The method of any one of claims 1 to 12, wherein R⁶ is OH.

15. The method of any one of claims 1 to 12, wherein R⁶ is OCH₃.

16. The method of any one of claims 1 to 15, wherein R⁷ is H.

17. The method of any one of claims 1 to 15, wherein R⁷ is OH.

18. The method of any one of claims 1 to 15, wherein R⁷ is OCH₃.

19. The method of any one of claims 1 to 18, wherein R⁸ is H.

20. The method of any one of claims 1 to 18, wherein R⁸ is OH.

21. The method of any one of claims 1 to 18, wherein R⁸ is OCH₃.

22. The method of claim 1, wherein the bitter-masking compound is selected from

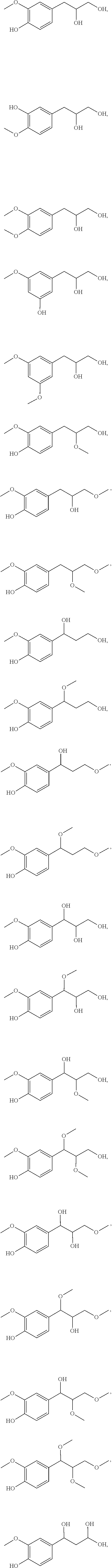

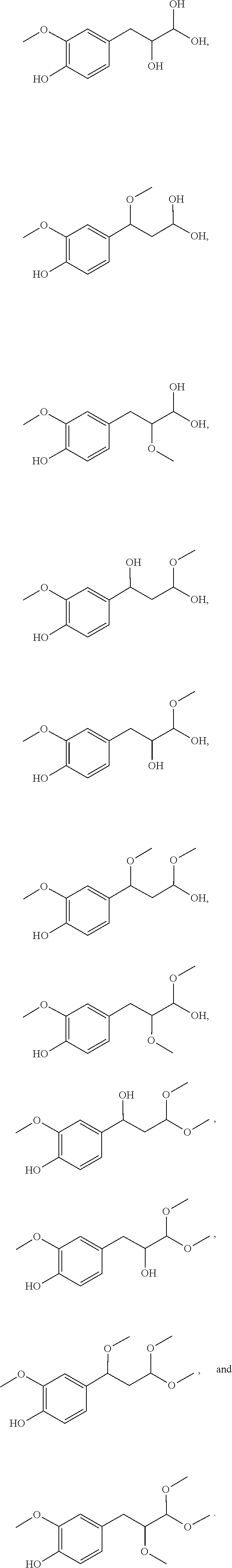

23. The method of claim 22, wherein the bitter-masking compound is selected from

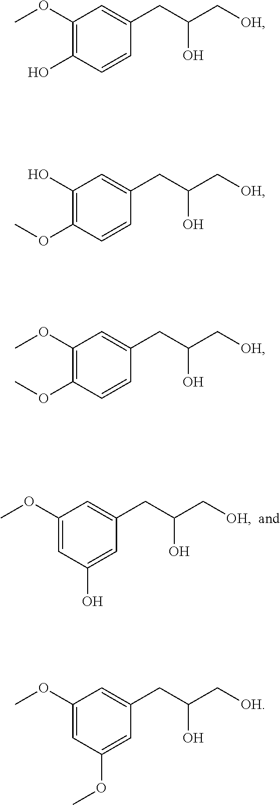

24. The method of claim 22, wherein the bitter-masking compound is selected from

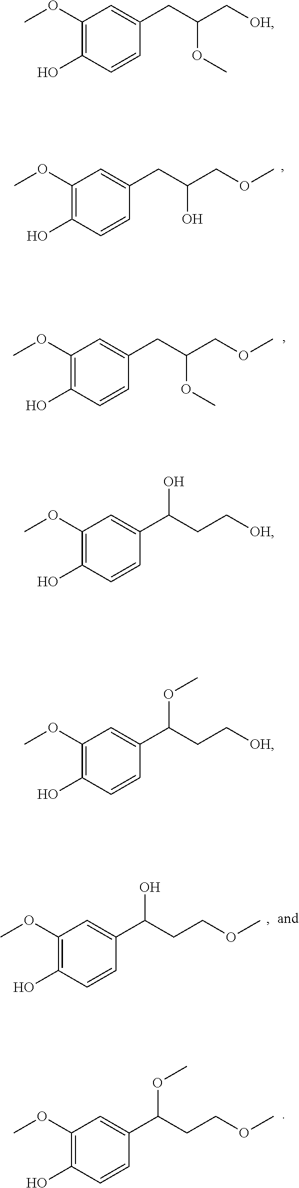

25. The method of claim 1, wherein the bitter-masking compound comprises 3-(4-hydroxy-3-methoxyphenyl) propane-1,2-diol.

26. The method of claim 1, wherein the bitter-masking compound comprises (2R)-3-(4-hydroxy-3-methoxyphenyl) propane-1,2-diol.

27. The method of claim 26, wherein (2R)-3-(4-hydroxy-3-methoxyphenyl) propane-1,2-diol is extracted from allspice.

28. The method of any one of claims 1 to 27, wherein the bitter-masking compound is added to the ingestible composition in an amount sufficient to reduce the perceived bitter taste of the ingestible composition.

29. The method of claim 28, wherein the amount of the bitter-masking compound sufficient to reduce the perceived bitter taste of the ingestible composition is from about 0.001 ppm to about 1000 ppm, based on the amount of the ingestible composition.

30. The method of any one of claims 1 to 29, wherein the ingestible composition contains a bitter substance selected from polyphenols, alkaloids, limonoids, medicaments, or a combination thereof.

31. The method of claim 30, wherein the bitter substance comprises a flavonoid, a tannin, a lignan, quinine, theobromine, limonin, linoleic acid, denatonium benzoate, amarogentin, a fluoroquinolone, aspirin, paracetamol, guaifenesin, ambroxol, salicin, an antibiotic, or a combination thereof.

32. The method of any one of claims 1 to 31, wherein the ingestible composition comprises a bitter substance that activates a TAS2R receptor expressed in oral tissue.

33. The method of claim 32, wherein the TAS2R receptor is selected from the group of TAS2R14, TAS2R10, TAS2R46, and a combination thereof.

34. The method of any one of claims 1 to 33, further comprising adding to the ingestible composition one or more secondary components selected from the group of oleic acid, β-cyclodextrin, sodium caseinate, homoeriodictyol, eriodictyol, sterubin, 6-methoxyflavanone, sodium ferulate salt, homoeriodictyol disodium salt, eriodictyol 7-methyl ether, N-(1-carboxyethyl)-3-hydroxy-6-hydroxymethyl pyridinium inner salt, N-carboxymethyl-3-hydroxy-6-hydroxymethyl-pyridinium inner salt, N-(1,3-dicarboxypropyl)-3-hydroxy-6-hydroxymethyl-pyridinium inner salt, (S)—N-(1-carboxyethyl)-3-hydroxy-6-hydroxy-methylpyridinium inner salt, (R)—N-(1-carboxyethyl)-3-hydroxy-6-hydroxy-methyl-pyridinium inner salt, (S)-N-[N-(1-carboxy-2-phenylethyl)-carboxamido-methyl]-3-hydroxy-6-hydroxymethyl-pyridinium inner salt, N-[1-[(3,5-dimethyl-4-isoxazolyl)methyl]-1H-pyrazol-4-yl]-α-methylbenzeneacetamide, N-[1-[(3,5-dimethyl-4-isoxazolyl)methyl]-1H-pyrazol-4-yl]-3-methoxybenzamide, and 4-[[[(4-methoxyphenyl)methyl](phenylmethyl)amino]sulfonyl]benzoic acid.

35. A method of obtaining (2R)-3-(4-hydroxy-3-methoxyphenyl) propane-1,2-diol from allspice, comprising:
(a) admixing allspice with an extraction solvent to form a crude mixture having a liquid phase and a solid phase, wherein the extraction solvent comprises a mixture of water and ethanol, containing from about 70 wt. % to about 90 wt. % ethanol based on the total weight of the extraction solvent; and
(b) collecting a first portion of the liquid phase, wherein the first portion of the liquid phase would elute from a liquid chromatography column using a first elution solution comprising water and ethanol containing about 20 wt. % to about 40 wt. % ethanol based on the total weight of the first elution solution,
wherein the first portion of the liquid phase comprises (2R)-3-(4-hydroxy-3-methoxyphenyl) propane-1,2-diol.

36. The method of claim 35, wherein step (b) comprises:
loading the liquid phase onto a chromatography column;
eluting the first portion of the liquid phase from the chromatography column using a first elution solution comprising water and ethanol, wherein the first elution solution comprises from about 20 wt. % to about 40 wt. % ethanol, based on the total weight of the first elution solution; and
collecting a first elution fraction comprising the first elution solution and the first portion of the liquid phase.

37. The method of claim 36, further comprising concentrating the liquid phase prior to loading the liquid phase onto the chromatography column.

38. The method of claim 35, further comprising:
(c) collecting a second portion of the liquid phase, wherein the second portion of the liquid phase would elute from a liquid chromatography column using a second elution solution comprising water and ethanol containing about 40 wt. % to about 60 wt. % ethanol, based on the total weight of the second elution solution,
wherein the second portion of the liquid phase comprises (2R)-3-(4-hydroxy-3-methoxyphenyl) propane-1,2-diol.

39. The method of claim 38, wherein step (c) comprises:
eluting the second portion of the liquid phase from a chromatography column using the second elution solution comprising water and ethanol, wherein the second elution solution comprises from about 40 wt. % to about 60 wt. % ethanol, based on the total weight of the second elution solution; and
collecting a second elution fraction comprising the second elution solution and the second portion of the liquid phase.

40. The method of claim 39, further comprising concentrating the liquid phase and loading the liquid phase onto the chromatography column.

41. The method of any one of claims 35 to 40, wherein allspice and the extraction solvent are admixed at a temperature in a range from about 20° C. to about 80° C.
